(19) United States
(12) Patent Application Publication
(10) Pub. No.: US 2010/0104669 A1
(43) Pub. Date: Apr. 29, 2010

(54) Method for preparing an aqueous solution containing triterpenic acid, aqueous solution containing triterpenic acid, and use thereof

(76) Inventors: Armin Scheffler, Niefern-Oeschelbronn (DE); Sebastian Jaeger, Wiernsheim-Pinache (DE); Markus Beffert, Pforzheim (DE); Katharina Hoppe, Kaempelbach (DE)

Correspondence Address: MUIRHEAD AND SATURNELLI, LLC, 200 FRIBERG PARKWAY, SUITE 1001, WESTBOROUGH, MA, 01581, US

(21) Appl. No.: 12/290,235

(22) Filed: Oct. 28, 2008

(51) Int. Cl. A61K 36/185; A61K 31/575

(52) U.S. Cl. 424/725; 514/182

(57) ABSTRACT

A method for preparing an aqueous solution containing triterpenic acid, an aqueous solution containing triterpenic acid, and use thereof, includes preparation of a basic aqueous solution containing triterpenic acid and reduction of the pH of the basic aqueous solution containing triterpenic acid by adding an acidic aqueous solution in the presence of a stabilizing additive. The system may also provide an aqueous physiological solution of a plant extract containing triterpenic acid, a method for preparing same, and use thereof for therapeutic purposes that includes aqueous physiological solutions rich in triterpenic acid which includes pharmacologically effective amounts of triterpenic acids.

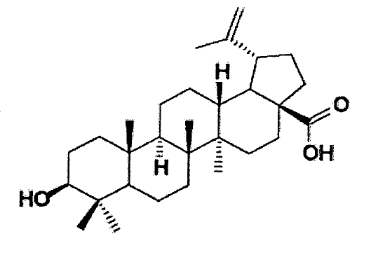

### TECHNICAL FIELD

This application relates to a method for preparing an aqueous solution containing triterpenic acid, an aqueous solution containing triterpenic acid, and use thereof.

### BACKGROUND OF THE INVENTION

Triterpenic acids are pharmacologically highly efficient active substances which are widespread in nature, primarily from plant sources. Triterpenic acids occur predominantly as pentacyclic compounds, which are formally derived from isoprene.

The bark of birch and sycamore trees contains, for example, betulinic acid as a pentacyclic triterpenoid, whose antitumoral activity has been described in numerous publications.

A method for extracting triterpenoids from plants or plant components is described, for example, in WO 2001/72315 A1 or WO 2004/016336 A1.

DE 100 15 353 A1 describes an emulsion which contains a plant extract, the plant extract containing at least one triterpenoid and/or a derivative of a triterpenoid. Such emulsions, which contain triterpenoids in a concentration of 3% to 15%, are obtained by dispersing a triterpenoid, obtained by extraction, in an oil or fat. The emulsion is used as a component of a salve, lotion, or cream for application to the skin, in particular for dermatological modifications in the skin.

Ursolic acid, which is likewise a pentacyclic triterpenic acid, may be obtained, for example, from apple cores, huckleberries, or lingonberries, and is known for its stimulation of the metabolism of the skin. Glycyrrhetic acid and its glycoside glycyrrhizic acid, which may be obtained from an extract of licorice (Glycyrrhiza alba), have anti-inflammatory and anticarcinogenic effects. Boswellic acid, which constitutes 50% of the resin of frankincense (olibanum), is used as a therapeutic agent in the treatment of rheumatism. Boswellic acid is also known for its analgesic and calmative effect. Oleanolic acid, which is likewise a pentacyclic triterpenic acid, is found in the leaves of the olive tree or ivy.

Oleanolic acid is the primary triterpenoid in mistletoe (Viscum album), in addition to betulinic acid and ursolic acid. The same as for betulinic acid, oleanolic acid and ursolic acid also have antitumoral activity.

Corosolic acid, which may be obtained as an extract from the leaves of the banaba tree (Lagerstroemia speciosa), has an effect which lowers the blood glucose level when administered orally.

In addition to its antitumoral activity, the triterpenic acid betulinic acid contained in birch and sycamore bark and mistletoe has an antiseptic effect, as well as an antiviral effect against HIV.

Betulinic acid is a pentacyclic triterpenic acid which belongs to the group of lupanes. The characteristic feature of the lupanes is a ring containing five carbon atoms within a pentacyclic system. The group feature of the oleananes and ursanes is a pentacyclic system composed only of rings containing six carbon atoms, the difference in ring E being the position of one methyl group. Oleanolic acid is an important representative of the oleananes, and ursolic acid belongs to the ursanes.

Betulinic acid is illustrated as an example of such an acid in the accompanying figure.

U.S. Pat. No. 6,482,857 B1, US 2003/0139471 A1, U.S. Pat. No. 6,451,777 B1, and US 2003/014526 A1 describe aqueous suspensions or emulsions of triterpenic acids for use as a hair tonic. WO 2006/088385 describes bioactive complexes of triterpenic acids for therapeutic use. Triterpene concentrates containing ursolic acid and oleanolic acid for use in foods are described in EP 1 250 852 B1.

The influence of cyclodextrins on the hydrophilicity of triterpenic acids is the subject of an article from J. Chromatogr. A, 1049 (2004), 37-42, ISSN: 0021-9673 (B. Claude et al.) and another article from J. Mass. Spectrom. 2003; 38: 723-731 (Guo et al.).

Triterpenic acids are distinguished by high thermal stability. The melting point of oleanolic acid (molecular weight: 456 g/mol) is above 300° C., that of betulinic acid (molecular weight: 456 g/mol) is between 275° C. and 278° C., and that of ursolic acid (molecular weight: 456 g/mol) is approximately 278° C.

Although the pharmacological activity of the triterpenoids is generally recognized, and adverse side effects from these natural substances have not been observed heretofore, further use of these active substances is limited primarily by their low solubility in water. All of the previously mentioned triterpenoids or extracts containing triterpenoids have the disadvantage that the active substances have insufficient solubility in an aqueous physiological system, so that for administration in the form of injection preparations, for example, it has not been possible thus far to obtain a sufficient quantity and concentration of active substance.

Triterpenic acids such as oleanolic acid, betulinic acid, or ursolic acid, for example, are soluble in pyridine and tetrahydrofuran, but only slightly soluble in dichloromethane, chloroform, and cold organic solvents, the solubility improving significantly with increasing temperature. These acids are practically insoluble in water, with a solubility of less 0.1 μg/mL.

Accordingly, it would be desirable to provide a method for preparing an aqueous solution containing triterpenic acid, an aqueous solution containing triterpenic acid, and a method for using such an aqueous solution.

### SUMMARY OF THE INVENTION

According to the system described herein, a method for preparing an aqueous solution containing triterpenic acid includes preparation of a basic aqueous solution containing triterpenic acid and reduction of the pH of the basic aqueous solution containing triterpenic acid by adding an acidic aqueous solution in the presence of a stabilizing additive. The stabilizing additive may be at least one of: a polysaccharide and an oligosaccharide, and the oligosaccharide may be a cyclodextrin. The pH of the basic aqueous solution containing triterpenic acid may initially be between 8 and 14 and may be reduced to a value between 5 and 9.

The preparation of the basic solution containing triterpenic acid may include providing a solid containing triterpenoid, and dissolving the solid containing triterpenoid in a basic aqueous medium at a temperature between 20° C. and 200° C., and may be at a pressure between 2 and 150 bar. Trisodium phosphate may be used to adjust the pH in the preparation step and phosphoric acid or citric acid may be used to reduce the pH in the reducing step. The stabilizing additive may be added to the acidic aqueous solution and/or may be added to the basic aqueous solution. At least one additional aqueous plant extract may be at least one of: added to the basic aqueous solution in the preparation step, added to the pH-reduced solution in sthe reducing step, and added while carrying out the reducing step. The additional aqueous plant extract may be a plant extract of a triterpenoid-containing plant or parts thereof. The additional aqueous plant extract may be a plant extract of at least one of: mistletoe, birch, sycamore, and parts thereof. The additional plant extract may have a pH between 5 and 9. The additional extract may be a plant extract containing vesicles.

According further to the system described herein, an aqueous solution of a plant extract contains at least one of: a triterpenic acid and a derivative of a triterpenic acid, the solution having a triterpenic acid content of greater than 1 μg/mL at a pH of 5 to 9 in the presence of an additive which stabilizes the aqueous physiological solution of triterpenic acids, the stabilizing additive being a polysaccharide. The triterpenic acid and/or the derivative of the triterpenic acid may include at least one of: asiatic acid, betulinic acid, boswellic acid, corosolic acid, glycyrrhetic acid, madecassic acid, oleanolic acid, phellonic acid and ursolic acid. The plant extract may be an extract of at least one of: mistletoe, birch, sycamore, other triterpenoid-containing plants, and parts thereof. Salt concentration in the solution may be in a range of 5-140 mM, and may be between 25 mM and 35 mM. A mistletoe extract containing vesicles may be included in the solution.

According further to the system described herein, a method is provided for using an aqueous solution for a therapeutic purpose, the solution including at least one of: a triterpenic acid and a derivative of a triterpenic acid, the solution having a triterpenic acid content of greater than 1 μg/mL at a pH of 5 to 9 in the presence of an additive which stabilizes the aqueous physiological solution of triterpenic acids, the stabilizing additive being a polysaccharide. The therapeutic purpose may include use as a food supplement, a physiologically injectable preparation, a physiologically injectable preparation for tumor therapy, a physiologically injectable preparation for hepatitis therapy, a therapeutic treatment of tumor diseases of the skin and a therapeutic agent against viruses and bacteria. The therapeutic purpose may also include use for flocculation stabilization of plant extracts, for producing a therapeutic agent, for producing a food supplement, for producing a physiologically injectable preparation, for producing a physiologically injectable preparation for tumor therapy, for producing a physiologically injectable preparation for hepatitis therapy, for producing a therapeutic agent for the therapeutic treatment of tumor diseases of the skin, and for producing a therapeutic agent for treatment against viruses and bacteria.

### BRIEF DESCRIPTION OF THE DRAWING

The sole FIGURE shows an illustration of betulinic acid as an example of a triterpenic acid for use in connection with the system described herein.

### DETAILED DESCRIPTION OF VARIOUS EMBODIMENTS

In the context of the system described herein, a solution containing triterpenic acid may be understood to be a solution which contains at least one triterpenic acid and/or one derivative of a triterpenic acid. An aqueous physiological solution may be understood below to mean an aqueous solution having a pH between 6 and 8.

Cyclic triterpenoids containing no more than two functional oxygen-rich groups are very slightly soluble in aqueous physiological solutions. Thus, for example, the solubility of betulinic acid in water is less than 0.1 μg/mL. According to the method described herein, the presence of a stabilizing additive allows the pH of the basic aqueous solution to be reduced to the pH of an aqueous physiological solution without precipitation of the triterpenic acids. In this manner an aqueous physiological solution is obtained in which therapeutically relevant quantities of at least one triterpenic acid or derivative thereof are present. The sole figure shows an illustration of betulinic acid as an example of a triterpenic acid for use in connection with the system described herein.

The basic solution containing triterpenic acid may be prepared in a customary manner by dissolving at least one triterpenic acid or derivative thereof in a suitable basic medium such as, for example, trisodium phosphate in water. Before dissolving, the triterpenic acid may be present in the form of a highly concentrated crystalline substrate which has been obtained beforehand by extraction from plants or plant components.

Also suitable, however, as a basic solution containing triterpenic acid is an extract solution obtained by extraction of triterpenic acids from plants or plant components. Any known method for extraction of triterpenoids from plants or plant components, using a basic extraction agent, is suitable for obtaining such an extract solution.

The triterpenic acids may be dissolved in the basic aqueous solution at temperatures between 20° C. and 200° C. and pressures between 5 bar and 150 bar.

The pH of the basic aqueous solution is adjusted to a value between 8 and 14, for example. Trisodium phosphate, for example, is suitable for adjusting the pH.

Physiologically acceptable acids such as phosphoric acid or citric acid, for example, are suitable for reducing the pH of the basic aqueous solution containing triterpenic acid to physiological values (pH between 6 and 8).

Polysaccharides in particular are suitable as stabilizing additives which prevent flocculation at reduced pH.

Mistletoe, birch, or sycamore, all of which contain high proportions of oleanolic acid or betulinic acid, are particularly well suited as plant raw materials for obtaining the at least one triterpenic acid contained in the aqueous solution. It is noted that triterpenoid-containing plants usually contain various triterpenoids, which are present in corresponding concentrations in the extract obtained by extraction of the plants. Thus, in addition to oleanolic acid and betulinic acid, mistletoe also contains low concentrations of ursolic acid, β-amyrin, lupeol, β-amyrin acetate, and lupeol acetate.

Depending on the extract used for preparing the basic solution containing triterpenoid, i.e., depending on the plant material used for obtaining the extract, different triterpenoids are present in different concentrations in this solution.

In particular, a polysaccharide corresponding to the particular plant material may be used as a stabilizing additive. Thus, for example, in obtaining an aqueous physiological mistletoe extract for stabilizing the triterpenic acids, a polysaccharide-containing extract from mistletoe berries may be used.

The salt concentration of the neutralized (physiological) solution containing triterpenic acid may be set to suitable desired values, for example 3 mM to 140 mM, preferably 10 mM. Depending on the intended administration, for a subcutaneous injection solution, for example, isotonization, for example also using saccharides, may be performed.

The method described above, using any given triterpenic acids, allows production of injectable therapeutic preparations containing triterpenic acid, and having a triterpenic acid content which is far above the solubility limit of triterpenes in water and having a triterpenoid content of greater than 0.5 μg/mL, preferably greater than 1 μg/mL.

In an embodiment of the system described herein, the aqueous physiological solutions containing triterpenic acid may be used not only as such, but may also be combined with other aqueous physiological plant extracts which do not contain triterpenoids but contain other active substances from corresponding plants. Additional active substances may be introduced into the aqueous physiological preparation by using this combination. One example of such an additional extract is mistletoe extract containing vesicles.

Surprisingly, it has been found that, when combined with other plant extracts, the solution containing triterpenic acid stabilizes the other plant extracts against flocculation. Since most aqueous plant extracts tend toward flocculation, this positive effect is particularly important for the use and storage of therapeutic plant extracts. A corresponding flocculation-stabilized extract contains, in addition to other active substances derived from plants, a therapeutically effective quantity of triterpenic acids.

The combination of an aqueous physiological solution containing triterpenic acid with an additional plant extract and the observed stabilization of the aqueous physiological solution containing multiple plant extracts against flocculation opens up unexpected therapeutic possibilities for the use of plant extracts. Thus, injectable preparations containing triterpenic acid may be formulated to which, in addition to the triterpenic acids, other active substances derived from plants are added in a targeted manner. These additional active substances derived from plants are not limited to triterpenoids, and may include any active substance which is obtainable as a plant extract.

The flocculation-stabilizing effect of the solution rich in triterpenic acid is based on initial tests on a combination of the triterpenic acids with the at least one stabilizer.

Embodiments of the system described herein are explained below with reference to examples, discussed as follows.

### EXAMPLE 1

Dried annual shoots of mistletoe were extracted with n-heptane at 120° C. and >2 bar pressure. The precipitate obtained upon cooling of the extraction agent was composed of more than 60% oleanolic acid and more than 3% betulinic acid. The triterpenic acid-rich powder thus obtained was extracted with water in the presence of trisodium phosphate at 120° C. and >2 bar pressure to produce a supersaturated basic solution.

The basic triterpenoid-rich solution, cooled to room temperature and filtered through a 0.22 μm filter, contained more than 80 μg/mL oleanolic acid and more than 8 μg/mL betulinic acid. The basic solution thus prepared was then adjusted to pH 7.4 using an aqueous phosphoric acid solution, in the presence of 1.6 μg/mL polysaccharides which had been obtained from mistletoe berries. The extent of dilution and the solvent quantities were selected in such a way that the salt concentration of the neutralized triterpenic acid-rich solution was between 5 and 140 mM.

### EXAMPLE 2

A portion of an aqueous physiological mistletoe extract rich in active substance but low in triterpenoid was mixed with a portion of an aqueous physiological solution of a mistletoe extract from Example 1.

This resulted in an aqueous physiological plant extract which was stabilized against flocculation and which in addition to the other water-extractable active substances of the mistletoe contained 90% of the triterpenic acids originating from the basic extract.

### EXAMPLE 3

Two parts of aqueous physiological mistletoe extract low in triterpenoid were each combined with one part of basic triterpenic acid solution and one part of acidic polysaccharide solution.

The same as in Example 2, this resulted in an aqueous physiological solution which in addition to the water-extractable active substances of the mistletoe contained therapeutically effective quantities of triterpenic acids. The plant extract thus prepared is stable against flocculation.

### EXAMPLE 4

The triterpenoid-rich powder described in Example 1 was extracted with water in the presence of trisodium phosphate (10 mM) and 2 mg/mL cyclodextrins at 120° C. and >2 bar.

The basic triterpenic acid-rich solution contained more than 100 μg/mL oleanolic acid and more than 8 μg/mL betulinic acid. The basic solution thus prepared was then adjusted to pH 7.4 using an aqueous phosphoric acid solution. The extent of dilution and the solvent quantities were selected in such away that the salt concentration of the neutralized triterpenic acid-rich solution was between 5 and 140 mM.

This aqueous physiological solution rich in triterpenic acid may be used as described in Example 2.

In addition, the basic solution containing cyclodextrins and triterpenic acids from this example may be used as described in Example 3.

### EXAMPLE 5

Polysaccharides obtained from mistletoe berries may be present during the neutralization described in Example 4.

### EXAMPLE 6

For the method described in Example 3 the triterpenic acid-rich solution containing cyclodextrin described in Example 4 may be used. The effects achieved are the same as those described in Example 3.

Other embodiments of the invention will be apparent to those skilled in the art from a consideration of the specification or practice of the invention disclosed herein. It is intended that the specification and examples be considered as exemplary only, with the true scope and spirit of the invention being indicated by the following claims.

What is claimed is:

1. A method for preparing an aqueous solution containing triterpenic acid, comprising:
a) preparing a basic aqueous solution containing triterpenic acid; and
b) neutralizing pH of the basic aqueous solution containing triterpenic acid by adding an acidic aqueous solution in the presence of a stabilizing additive.

2. The method as recited in claim 1, wherein the stabilizing additive is at least one of: a polysaccharide and an oligosaccharide.

3. The method as recited in claim 2, wherein the oligosaccharide is a cyclodextrin.

4. The method as recited in claim 1, wherein the pH of the basic aqueous solution containing triterpenic acid in step a) is between 8 and 14.

5. The method as recited in claim 1, wherein the pH of the solution in step b) is reduced to a value between 5 and 9.

6. The method as recited in claim 1, wherein the preparation of the basic solution containing triterpenic acid includes:
a1) providing a solid containing triterpenoid; and
a2) dissolving the solid containing triterpenoid in a basic aqueous medium at a temperature between 20° C. and 200° C.

7. The method as recited in claim 6, wherein the pressure in step a) is between 2 and 150 bar.

8. The method as recited in claim 1, wherein trisodium phosphate is used to adjust the pH in step a).

9. The method as recited in claim 1, wherein phosphoric acid or citric acid is used to reduce the pH in step b).

10. The method as recited in claim 1, wherein the stabilizing additive is added to the acidic aqueous solution.

11. The method as recited in claim 1, wherein the stabilizing additive is added to the basic aqueous solution.

12. The method as recited in claim 1, wherein at least one additional aqueous plant extract is at least one of: added to the basic aqueous solution in step a), added to the pH-reduced solution in step b), and added while carrying out step b).

13. The method as recited in claim 12, wherein the at least one additional aqueous plant extract is a plant extract of a triterpenoid-containing plant or parts thereof.

14. The method as recited in claim 13, wherein the at least one additional aqueous plant extract is a plant extract of at least one of: mistletoe, birch, sycamore, and parts thereof.

15. The method as recited in one of claim 12, wherein the at least one additional plant extract has a pH between 5 and 9.

16. The method as recited in one of claim 12, wherein the at least one additional extract is a plant extract containing vesicles.

17. An aqueous solution of a plant extract, comprising:
at least one of: a triterpenic acid and a derivative of a triterpenic acid, the solution having a triterpenic acid content of greater than 1 pg/mL at a pH of 5 to 9 in the presence of an additive which stabilizes the aqueous physiological solution of triterpenic acids, the stabilizing additive being a polysaccharide.

18. The solution as recited in claim 17, wherein the at least of: the triterpenic acid and the derivative of the triterpenic acid includes at least one of: asiatic acid, betulinic acid, boswellic acid, corosolic acid, glycyrrhetic acid, madecassic acid, oleanolic acid, phellonic acid and ursolic acid.

19. The solution as recited in claim 17, wherein the plant extract is an extract of at least one of: mistletoe, birch, sycamore, other triterpenoid-containing plants, and parts thereof.

20. The solution as recited in claim 17, wherein salt concentration in the solution is 5-140 mM.

21. The solution as recited in claim 20, wherein the salt concentration is between 25 mM and 35 mM.

22. The solution as recited in claim 17, further comprising a mistletoe extract containing vesicles.

23. A method of using an aqueous solution for a therapeutic purpose, the solution including at least one of: a triterpenic acid and a derivative of a triterpenic acid, the solution having a triterpenic acid content of greater than 1 μg/mL at a pH of 5 to 9 in the presence of an additive which stabilizes the aqueous physiological solution of triterpenic acids, the stabilizing additive being a polysaccharide.

24. The method of claim 23, wherein the therapeutic purpose includes use as a food supplement.

25. The method of claim 23, wherein the therapeutic purpose includes use as a physiologically injectable preparation.

26. The method of claim 23, wherein the therapeutic purpose includes use as a physiologically injectable preparation for tumor therapy.

27. The method of claim 23, wherein the therapeutic purpose includes use as a physiologically injectable preparation for hepatitis therapy.

28. The method of claim 23, wherein the therapeutic purpose includes use as a therapeutic treatment of tumor diseases of the skin.

29. The method of claim 23, wherein the therapeutic purpose includes use as a therapeutic agent against viruses and bacteria.

30. The method of claim 23, wherein the therapeutic purpose includes use for flocculation stabilization of plant extracts.

31. The method of claim 23, wherein the therapeutic purpose includes use for producing a therapeutic agent.

32. The method of claim 23, wherein the therapeutic purpose includes use for producing a food supplement.

33. The method of claim 23, wherein the therapeutic purpose includes use for producing a physiologically injectable preparation.

34. The method of claim 23, wherein the therapeutic purpose includes use for producing a physiologically injectable preparation for tumor therapy.

35. The method of claim 23, wherein the therapeutic purpose includes use for producing a physiologically injectable preparation for hepatitis therapy.

36. The method of claim 23, wherein the therapeutic purpose includes use for producing a therapeutic agent for the therapeutic treatment of tumor diseases of the skin.

37. The method of claim 23, wherein the therapeutic purpose includes use for producing a therapeutic agent for treatment against viruses and bacteria.
